(19) United States
(12) Patent Application Publication
(10) Pub. No.: US 2025/0230130 A1
(43) Pub. Date: Jul. 17, 2025

(54) NOVEL STABILIZED IMIDAZOLINE COMPLEXES

(71) Applicant: Baker Hughes Oilfield Operations LLC, Houston, TX (US)

(72) Inventors: Tracey Jackson, Richmond, TX (US); Peng Jin, Sugar Land, TX (US); Oleg A. Mazyar, Katy, TX (US); Shujun Gao, Katy, TX (US)

(73) Assignee: Baker Hughes Oilfield Operations LLC, Houston, TX (US)

(21) Appl. No.: 18/411,692

(22) Filed: Jan. 12, 2024

(51) Int. Cl. C07D 233/10

(52) U.S. Cl. CPC C07D 233/10

(57) ABSTRACT

A stabilized imidazoline complex includes a proton-stabilizing molecule and a positively charged imidazoline compound, where the proton-stabilizing molecule has a first lone-pair-bearing atom and a second lone-pair-bearing atom. The proton-stabilizing molecule is a dye. The positively charged imidazoline compound includes an imidazoline ring and a hydrogen atom, and a covalent bond connects the hydrogen atom with a nitrogen atom of the imidazoline ring. At least one hydrogen bond connects the hydrogen atom with the first lone-pair-bearing molecule and the second lone-pair-bearing atom of the proton-stabilizing molecule.

### FIELD OF THE INVENTION

The present application is generally directed at chemically stabilized compounds, and in particular to novel stabilized imidazoline complexes and methods for preparing the same.

### BACKGROUND OF THE INVENTION

Imidazoline compounds serve a variety of industrial and commercial applications. For example, various corrosion inhibitors have been developed to combat metal corrosion of equipment in industries including, but not limited to, the oil and gas industry. Among these, imidazolines are one of the most widely used and effective corrosion inhibitors. Imidazoline-based corrosion inhibitors are also soluble in a wide range of hydrocarbon and water-alcohol-based solutions. Based on continued demand for corrosion inhibitors, it is desirable to develop chemistries with new synergistic advantages. It is further desirable to develop corrosion inhibitors that are more easily detectable via chemical residual measurements.

In addition to their well-established use as corrosion inhibitors, imidazoline compounds have also been used as surfactants for a wide range of industrial applications. Imidazolines also find utility in many medications, including those for treating high blood pressure and nasal congestion.

Although widely adopted, there remains a need for improved imidazoline-based complexes that provide new synergies in a variety of applications, including but not limited to, corrosion inhibition, surfactant use, and drug development.

### SUMMARY OF THE INVENTION

In one aspect, a stabilized imidazoline complex is disclosed, where the stabilized imidazoline complex includes a proton-stabilizing molecule and a positively charged imidazoline compound, where the proton-stabilizing molecule is a dye. At least one hydrogen bond connects the positively charged imidazoline compound with a first lone-pair-bearing atom of the proton-stabilizing molecule.

In another aspect, a stabilized imidazoline complex includes a proton-stabilizing molecule and a positively charged imidazoline compound, where the proton-stabilizing molecule has a sulfur-based fragment of the formula O═S(═O)[R₁][R₂], where R₁ is a first portion of the proton-stabilizing molecule that is bonded with the sulfur (S) of the sulfur-based fragment, and where R₂ is a second portion of the proton-stabilizing molecule that is bonded with the sulfur (S). The positively charged imidazoline compound includes an imidazoline ring and a hydrogen atom, which is connected with a nitrogen atom of the imidazoline ring by a covalent bond. Further, at least one hydrogen bond connects the hydrogen atom with the first oxygen (O) of the sulfur-based fragment and the second oxygen (O) of the sulfur-based fragment.

In another aspect, a method of preparing a stabilized imidazoline complex is disclosed, where the method includes the steps of obtaining a positively charged imidazoline compound and mixing the positively charged imidazoline compound with a proton-stabilizing molecule, which has a first lone-pair-bearing atom and a second lone-pair-bearing atom. The proton-stabilizing molecule has a substructure of SSS═(O═S1(═O)CCCCO1.

### BRIEF DESCRIPTION OF THE DRAWINGS

The above and other objects and advantages of this invention may be more clearly seen when viewed in conjunction with the accompanying drawings wherein:

FIG. 1 depicts an exemplary embodiment of a stabilized imidazoline complex.

FIG. 2 depicts the comparative corrosion rate results for various dyes when tested in the absence of imidazoline.

FIG. 3 depicts the corrosion rate results for the dyes of FIG. 2 when combined with imidazoline.

### DETAILED DESCRIPTION

It has been discovered that certain molecules containing one or more atoms with unshared valence electrons have a particularly strong affinity for protonated imidazoline, and complexation of these molecules with protonated imidazoline causes dramatic changes in aqueous solubility and corrosion inhibitor performance for the latter. These complexes are stable in solution and favor strong association rather than moderate dissociation in equilibrium as most researchers believe. In other words, this complexation runs contrary to the popular belief that imidazoline does not form complexes with other molecules in the bulk. Besides achieving synergistic corrosion inhibiting performance, some of the resulting complexes are also highly active in the ultraviolet-visible spectrum and, therefore, are more easily detectable via chemical residual measurements. Further, these complexes are so stable that their Raman spectra in the presence of metal nanoparticles are different from that of their un-complexed components.

In one aspect, a stabilized imidazoline complex includes a proton-stabilizing molecule and a positively charged imidazoline compound.

The proton-stabilizing molecule component of the stabilized imidazoline complex has a structure that includes a first lone-pair-bearing atom. In some embodiments, the proton-stabilizing molecule component further includes a second lone-pair-bearing atom. By “lone-pair-bearing,” it is meant that the atom has one or more pairs of valence electrons that are not shared with another atom in a covalent bond. In certain embodiments, the first lone-pair-bearing atom and the second lone-pair-bearing atom are the same element; in others, they are different elements. In various non-limiting embodiments, the first and the second lone-pair-bearing atoms may be oxygen, nitrogen, fluorine, or sulfur.

In various embodiments, the proton-stabilizing molecule includes a sulfur-based fragment. By “fragment” is meant a substructure of the overall molecule that includes only some of the atoms and bonds for said overall molecule. In several embodiments, the sulfur-based fragment is of the formula O═S(═O)[R₁][R₂], where R₁ is a first portion of the proton-stabilizing molecule that is bonded with sulfur (S) of the sulfur-based fragment, and R₂ is a second portion of the proton-stabilizing molecule that is bonded with the same. In various embodiments the R₁ and R₂ portions have either the same or different elements and arrangements and quantities of atoms, bonds, and functional groups. By way of non-limiting example, the R₁ portion, the R₂ portion, or both may be an alkyl group. In certain embodiments, the sulfur-based fragment is a sulfuryl group; in other embodiments, the sulfur-based fragment is a sulfonyl group, and the proton-stabilizing molecule may be, for example, a sulfone or a sulfonyl halide. The first lone-pair-bearing atom and the second lone-pair-bearing atom are independently either one of the oxygens from the sulfur-based fragment or one of the atoms from the R₁ and R₂ portions of the proton-stabilizing molecule.

In various embodiments, the proton-stabilizing molecule is a dye. A non-limiting list of suitable dyes includes phenol red, pyrocatechol violet, eriochrome cyanine R, cresol red, chlorophenol red, bromocresol purple, bromophenol blue, bromothymol blue sodium salt, xylenol orange tetrasodium salt, xylenol orange disodium salt, methylthymol blue sodium salt, bromothymol blue, bromocresol green, and m-cresol purple. The structures of these exemplary dyes are depicted in Table 1.

TABLE 1

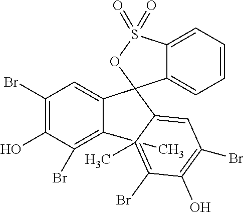

Bromocresol Green

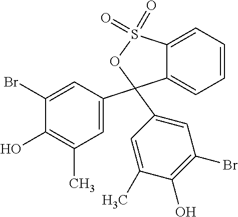 [SMILES: Cc1cc(C2(c3cc(C)c(O)c(Br)c3)OS(=O)(=O)c3ccccc32)cc(Br)c1O]

Bromocresol Purple

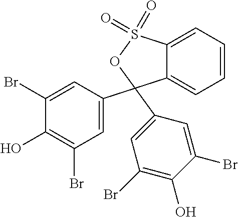 [SMILES: O=S1(=O)OC(c2cc(Br)c(O)c(Br)c2)(c2cc(Br)c(O)c(Br)c2)c2ccccc21]

Bromophenol Blue

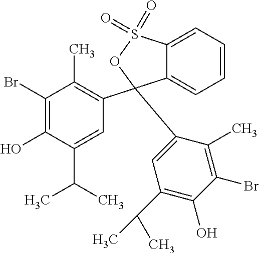 [SMILES: Cc1c(C2(c3cc(C(C)C)c(O)c(Br)c3C)OS(=O)(=O)c3ccccc32)cc(C(C)C)c(O)c1Br]

Bromothymol Blue

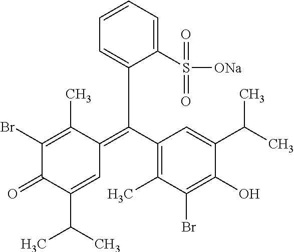

Bromothymol Blue Sodium Salt

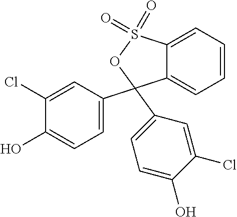 [SMILES: O=S1(=O)OC(c2ccc(O)c(Cl)c2)(c2ccc(O)c(Cl)c2)c2ccccc21]

Chlorophenol Red

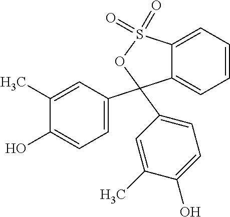 [SMILES: Cc1cc(C2(c3ccc(O)c(C)c3)OS(=O)(=O)c3ccccc32)ccc1O]

Cresol Red

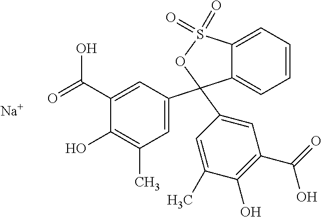 [SMILES: [Na+] ; Cc1cc(C2(c3cc(C)c(O)c(C(=O)O)c3)OS(=O)(=O)c3ccccc32)cc(C(=O)O)c1O]

Eriochrome Cyanine R

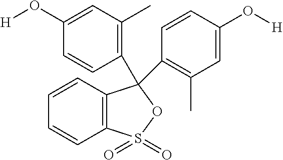 [SMILES: Cc1cc(O)ccc1C1(c2ccc(O)cc2C)OS(=O)(=O)c2ccccc21]

m-Cresol Purple

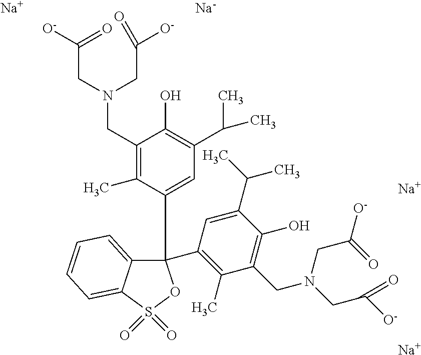

Methylthymol Blue Sodium Salt

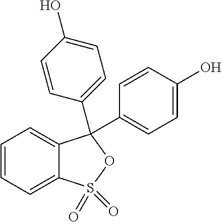 [SMILES: O=S1(=O)OC(c2ccc(O)cc2)(c2ccc(O)cc2)c2ccccc21]

Phenol Red

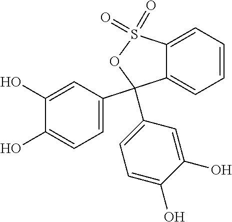 [SMILES: O=S1(=O)OC(c2ccc(O)c(O)c2)(c2ccc(O)c(O)c2)c2ccccc21]

Pyrocatechol Violet

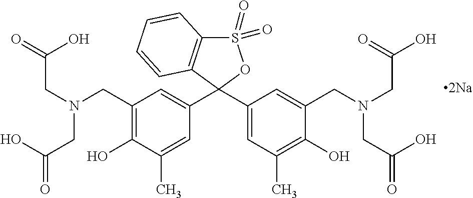 [SMILES: Cc1cc(C2(c3cc(C)c(O)c(CN(CC(=O)O)CC(=O)O)c3)OS(=O)(=O)c3ccccc32)cc(CN(CC(=O)O)CC(=O)O)c1O]

Xylenol Orange Disodium Salt

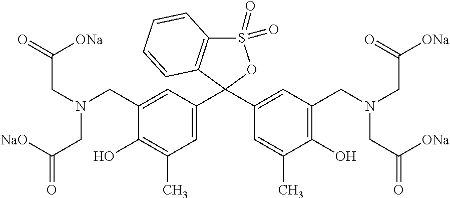

Xylenol Orange Tetrasodium Salt

The structure of the positively charged imidazoline compound component includes an imidazoline ring with two nitrogen atoms and a hydrogen atom connected to one of these nitrogen atoms by a covalent bond. The positively charged imidazoline compound includes an imine center and is either a 2-imidazoline isomer or a 3-imidazoline isomer. For example, suitable imidazolines may be derived from heavy polyamines, which are mixtures of higher molecular weight ethyleneamines of the formula H₂N(CH₂CH₂NH)ₙCH₂CH₂NH₂ where n is between 2 to 7. In various embodiments, the positively charged imidazoline compound is derived from the reaction of adiethylenetriamine (DETA)- or aminoethylethanolamine (AEEA)-base compound with a fatty acid, where suitable fatty acids include but are not necessarily limited to tall oil, coconut oil, soy oil, peanut oil, and other organic fatty acids with carbon chain lengths from C6 to C24. In one embodiment, the positively charged imidazoline compound is an imidazoline-based drug, such as moxonidine or oxymetazoline, which is presented as oxymetazoline hydrochloride in nasal decongestant sprays like Afrin® offered by Bayer Consumer Health.

Further, in certain embodiments, the positively charged imidazoline compound is a quaternary ammonium cation. The positively charged imidazoline compound may have the formula [R₃]N⁺(H2)[R₄], wherein R₃ is a portion of the imidazoline ring that is bonded with a positively charged nitrogen (N⁺ in the listed formula), and wherein R₄ is another portion of the imidazoline ring that is bonded with the positively charged nitrogen. By “portion” is meant a substructure of an overall molecule that includes only some of the atoms and bonds for the overall molecule. In certain embodiments, R₃ includes two atoms of the imidazoline ring on one side of the N⁺, and R₄ includes two different atoms of the imidazoline ring on the other side of the N⁺. It will be appreciated that in various embodiments the R₃ and R₄ portions include the same elements (e.g., carbon, hydrogen, nitrogen) and arrangements and quantities of atoms, bonds, and functional groups from one another. In other embodiments, R₃ and R₄ include different elements and arrangements and quantities of atoms, bonds, and functional groups.

It will be appreciated that the performance of the positively charged imidazoline compound can be tuned for various environments by using different proton-stabilizing molecules to form the stabilized imidazoline complex. Changing the structure of the proton-stabilizing molecule will likely change, for example, how the stabilized imidazoline complex aggregates in solution and on metal surfaces.

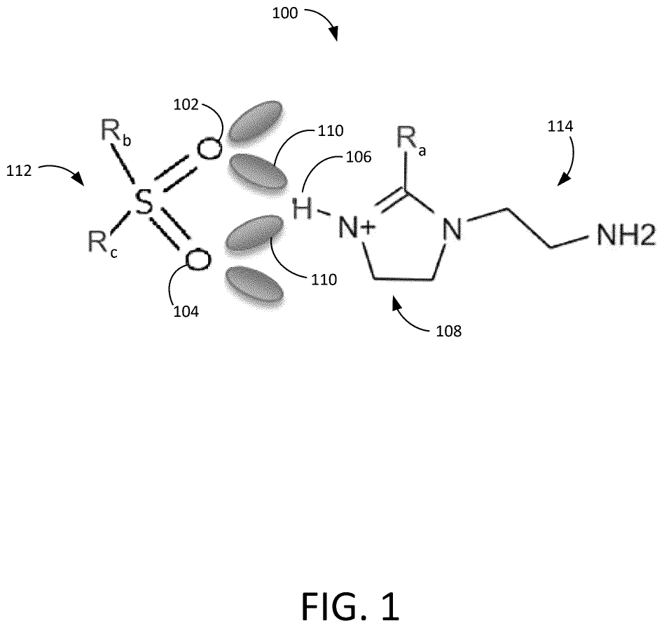

The stabilized imidazoline complex is generally formed through chemical association of the positively charged imidazoline compound component with the proton-stabilizing molecule component. The chemical association between these components may involve one or more intermolecular forces, including but not limited to hydrogen bonding and Van der Waals forces. In several embodiments, one or more hydrogen bonds connect the first lone-pair-bearing atom and the second lone-pair-bearing atom with the hydrogen atom of the positively charged imidazoline compound. FIG. 1 depicts an exemplary embodiment of the stabilized imidazoline complex where hydrogen bonding is accomplished via the unshared valence electrons of the adjacent oxygens; the exemplary “Rₐ” group depicted in FIG. 1 is an alkyl group of C8 to C22. In the depicted embodiment, the stabilized imidazoline complex 100 coordinates the first lone-pair-bearing atom 102 and the second lone-pair-bearing atom 104 (two oxygen atoms) to chelate the proton 106 of the imidazoline ring 108 by unshared valence electrons 110 of the sulfur fragment 112, where for the exemplary “R_{b}” and “R_{c}” groups, R_{b} is a first portion of the proton-stabilizing molecule that is bonded with sulfur (S) of the sulfur-based fragment, and R_{c} is a second portion of the proton-stabilizing molecule that is bonded with the same. The proton-stabilizing molecule therefore forms very strong associations with protonated imidazoline 114 because this oxygen pair structure allows the lone pairs 110 on the lone-pair bearing atoms (oxygen in this case) 102, 104 to lock the proton 106 between the imidazoline ring 108 and the sulfur fragment 112.

Because of equilibrium processes there will always be a little positively charged imidazoline compound present in aqueous solution at all pH levels. When the proton-stabilizing molecule is added, the association shifts the equilibrium by removing all of the positively charged imidazoline compound from solution. Due to LeChatelier's Principle, more positively charged imidazoline compound is then formed and subsequently removed until all imidazoline is converted to the stabilized imidazoline complex. This process proceeds until the limiting reagent is depleted. Depending on the components used, the stabilized imidazoline complex may experience one or more of the following observed changes: the stabilized imidazoline complex may become completely oil soluble, perform better as a corrosion inhibitor than acetate-salted imidazoline, be stable at pH 14, form imidazoline solids (thereby permitting their removal from solution), have improved stability at higher temperatures, have better chemical compatibility with high calcium brines, and have better corrosion inhibition performance in high calcium brines.

It is anticipated that the synergy achieved by the proton-stabilizing molecule and the positively charged imidazoline compound results, at least in part, from overlapping pi orbitals. The nitrogen and carbon atoms in the imine bond of the positively charged imidazoline compound 114 have pi orbitals, while the hydrogen on the imidazoline ring has a pseudo-pi orbital. These pi orbitals conduct electrons to the first and second lone-pair-bearing atoms of the proton-stabilizing molecule, creating a stabilized pi orbital structure that has improved synergistic performance interfacing with d orbitals of other molecules, including but not limited to molecules having groups of transition metal atoms (groups 3 through 12 on the periodic table).

In some embodiments, the stabilized imidazoline complex is obtained through a stepwise preparation method. To obtain the positively charged imidazoline compound component for the stabilized imidazoline complex, it may be necessary to protonate an unprotonated imidazoline with, for example, protonated water (H₃O⁺). The positively charged imidazoline compound, once obtained, is mixed with the proton-stabilizing molecule. Because some complexes may be more preferred than others when mixed, the order of addition plays a significant role in determining the structure of the resulting stabilized imidazoline complex. In some embodiments, the positively charged imidazoline compound is gradually added to the proton-stabilizing molecule. In other embodiments, the proton-stabilizing molecule is gradually added to the positively charged imidazoline compound.

The preparation method for the stabilized imidazoline complex may also include a pH modification step to promote chemical association between the positively charged imidazoline compound and the proton-stabilizing molecule. In one embodiment, a pH modifier is added in this step to reduce the pH level.

It will be appreciated that the proton-stabilizing molecule may be dissolved in a suitable solvent. Solvent choice and composition will have a significant impact on the structure and/or stability of the stabilized imidazoline complex. A suitable solvent is defined as one that will not disrupt complexing between the positively charged imidazoline compound and the proton-stabilizing molecule and will achieve solubility. In some embodiments, solvents such as methanol and water may not be suitable because they will disrupt complexing between a specific embodiment of the positively charged imidazoline compound and the proton-stabilizing molecule. However, in other embodiments, these solvents may not disrupt complexing and, therefore, be suitable. In some embodiments, one or more surfactants are introduced to the solvent to enable or improve solubility.

In various embodiments, the stabilized imidazoline complex is prepared in a blend with one or more inhibitors to further improve performance. Suitable inhibitors include but are not necessarily limited to primary amines, secondary amines, tertiary amines, quaternary amines, sulfur compounds (e.g., 2-mercaptoethanol, thioglycolic acid), phosphate esters, ethoxylated imidazolines, alkylpyridines, and surfactants.

### Example I

Complexation of imidazoline compounds with a proton-stabilizing molecule was observed when trying to separate out the latter in a brine stream containing high amounts of imidazoline compound relative to the proton-stabilizing molecule. Using Raman spectroscopy, it was discovered that proton-stabilizing molecules have a vastly different calibration curve when raw compared when formulated with the imidazoline compound.

Upon further investigation, it was discovered that imidazoline compounds formed stable complexes with various dyes, which were tested previously and confirmed to show no inhibition performance individually in the absence of imidazoline. The test conditions for these studies are shown in Table 2.

TABLE 2

| | |
|---|---|
| Test Type | Kettle |
| Temperature (° F.) | 150 |
| Brine (%) | 90 |
| Oil (%) | 10 Isopar |
| Stir Rate (rpm) | 150 |
| Total Pressure (psi) | 14.7 |
| Purge Brine with: | CO₂ sparging |
| Test Duration: | 18 hours |
| Corrosion Inhibitor based on: | Total Volume |
| Total Volume of Each Test Cell (mL) | 1,000 |
| Coupon Metallurgy | CS1018 |
| Coupon Prep Method | Media Blast |
| Coupon Size | Long |

The brine composition for these studies is shown in Table 3.

TABLE 3

| | Ion (ppm) | Salt | Calculated Grams |
|---|---|---|---|
| Na⁺ | N/A | NaCl | 941.22 |
| Mg²⁺ | 223 | MgCl₂ · 6 H₂O | 18.65 |
| Ca²⁺ | 1,115 | CaCl₂ · 2 H₂O | 40.90 |
| Cl⁻ | 59,720 | — | — |

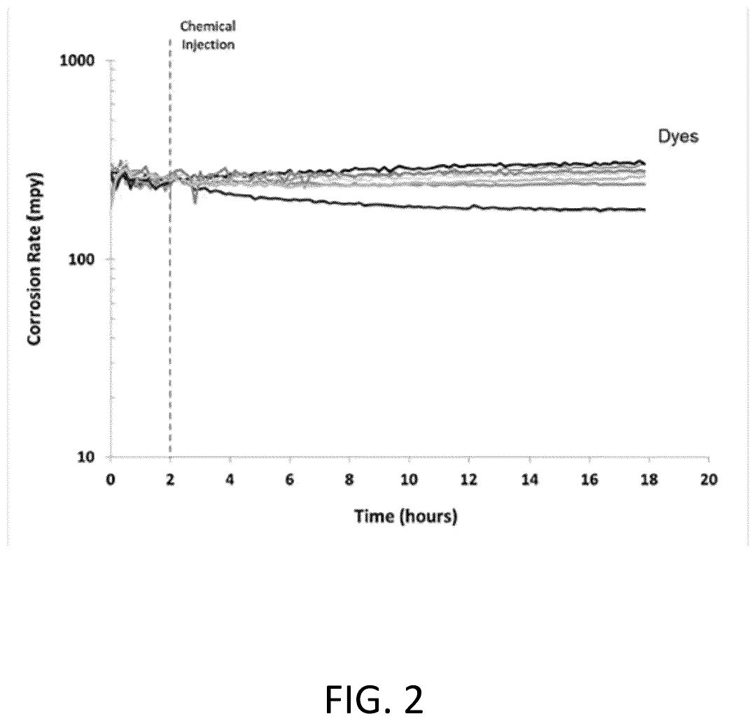

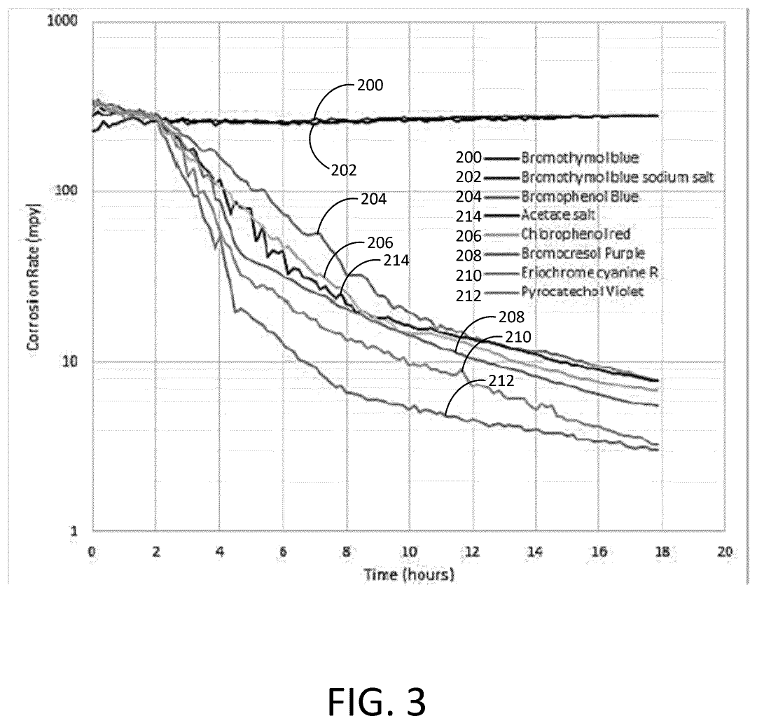

These dyes included bromothymol blue 200, bromothymol blue sodium salt 202, bromophenol blue 204, chlorophenol red 206, bromocresol purple 208, eriochrome cyanine R 210, and pyrocatechol violet 212. FIG. 2 shows the comparative corrosion rate results of these dyes and acetate salt 214 when tested at 10 ppm active component in the absence of imidazoline. Phenol red, cresol red, xylenol orange tetrasodium salt, xylenol orange disodium salt, and methylthymol blue sodium salt were not studied in this Example, due to unavailability of a suitable solvent. Due to the common structure of these dyes, it is anticipated that the untested dyes would perform similarly to those tested. FIG. 3 depicts the results of combining these dyes with imidazoline. The dyes were compared to an imidazoline-acetate formulation in the following amounts: 55 wt. % imidazoline, 20 wt. % dye, 12.5 wt. % water, and 12.5 wt. % isopropyl alcohol. However, due to the poor solubility of the dyes in this solvent package the dyes were only added at 10 wt. % (8 wt. % for bromophenol blue) and the solvent package was changed (i.e., 55 wt. % imidazoline, 8 or 10 wt. % dye, and 35 wt. % solvent as described below). The dyes were dissolved in a solvent consisting of 55 wt. % cyclohexanone, 20 wt. % ethanol, 20 wt. % isopropanol, and 5 wt. % water. The dye-imidazoline solutions were dosed at 30 ppm for each formulated solution. It was noted that, interestingly, the bromothymol variants made the imidazoline completely water insoluble. It is also noteworthy that none of these dyes are considered acids or bases so they did not contribute to the protonation or deprotonation of the imidazoline, yet each modified the performance of the imidazoline when compared to the imidazoline-acetate salt. These observations also imply that the association of protonated imidazoline and its proton-stabilizing partner forms a hydrogen bonded complex rather than the traditional “salt”.

In the foregoing specification, the invention has been described with reference to specific embodiments thereof. However, it will be evident that various modifications and changes can be made thereto without departing from the broader scope of the invention as set forth in the appended claims. Accordingly, the specification is to be regarded in an illustrative rather than a restrictive sense. For example, different stabilized imidazoline complexes, proton-stabilizing molecules, positively charged imidazoline compounds, fragments, lone-pair-bearing atoms, preparation steps, pH modifiers, solvents, proportions, dosages, and amounts not specifically identified or described in this disclosure or not evaluated in a particular Example are still expected to be within the scope of this invention.

The present invention may suitably comprise, consist of, or consist essentially of the elements disclosed and may be practiced in the absence of an element not disclosed. As used herein, the singular forms “a,” “an,” and “the” are intended to include the plural forms as well, unless the context clearly indicates otherwise. As used herein, the term “about” in reference to a given parameter is inclusive of the stated value and has the meaning dictated by the context (e.g., it includes the degree of error associated with measurement of the given parameter). As used herein, the term “and/or” includes any and all combinations of one or more of the associated listed items.

It is claimed:

1. A stabilized imidazoline complex comprising:
a proton-stabilizing molecule comprising a first lone-pair-bearing atom, wherein the proton-stabilizing molecule is a dye; and
a positively charged imidazoline compound, wherein at least one hydrogen bond connects the positively charged imidazoline compound with the first lone-pair-bearing atom.

2. The stabilized imidazoline complex of claim 1, wherein the proton-stabilizing molecule comprises a sulfur-based fragment of the formula O═S(═O)[R₁][R₂], wherein R₁ is a first portion of the proton-stabilizing molecule that is bonded with the sulfur (S) of the sulfur-based fragment, and wherein R₂ is a second portion of the proton-stabilizing molecule that is bonded with the sulfur (S).

3. The stabilized imidazoline complex of claim 2, wherein the first oxygen (O) of the sulfur-based fragment is the first lone-pair-bearing atom.

4. The stabilized imidazoline complex of claim 2, wherein the proton-stabilizing molecule further comprises a second lone-pair-bearing atom.

5. The stabilized imidazoline complex of claim 4, wherein the second oxygen (O) of the sulfur-based fragment is the second lone-pair-bearing atom.

6. The stabilized imidazoline complex of claim 1, wherein the proton-stabilizing molecule has a substructure of SSS═(O═S1(═O)CCCCO1).

7. The stabilized imidazoline complex of claim 1, wherein the proton-stabilizing molecule is a dye selected from the group consisting of phenol red, pyrocatechol violet, eriochrome cyanine R, cresol red, chlorophenol red, bromocresol purple, bromophenol blue, bromothymol blue sodium salt, xylenol orange tetrasodium salt, xylenol orange disodium salt, methylthymol blue sodium salt, bromothymol blue, bromocresol green, and m-cresol purple.

8. The stabilized imidazoline complex of claim 1, wherein the positively charged imidazoline compound comprises an imine center.

9. The stabilized imidazoline complex of claim 1, wherein the positively charged imidazoline compound is a quaternary ammonium cation.

10. The stabilized imidazoline complex of claim 1, wherein the positively charged imidazoline compound has the formula [R₃]N⁺(H2)[R₄], wherein R₃ is a first portion of the imidazoline that is bonded with the positively charged nitrogen (N⁺) of the formula, and wherein R₄ is a second portion of the imidazoline that is bonded with the positively charged nitrogen atom (N⁺).

11. The stabilized imidazoline complex of claim 1, wherein the positively charged imidazoline compound is derived from the reaction of a diethylenetriamine (DETA)- or aminoethylethanolamine (AEEA)-base compound with a fatty acid.

12. The stabilized imidazoline complex of claim 1, wherein the positively charged imidazoline compound is an imidazoline-based drug selected from the group consisting of moxonidine and oxymetazoline.

13. A stabilized imidazoline complex comprising:
a proton-stabilizing molecule comprising a sulfur-based fragment of the formula O═S(═O)[R₁][R₂], wherein R₁ is a first portion of the proton-stabilizing molecule that is bonded with the sulfur (S) of the sulfur-based fragment, wherein R₂ is a second portion of the proton-stabilizing molecule that is bonded with the sulfur (S); and
a positively charged imidazoline compound comprising an imidazoline ring and a hydrogen atom, wherein a covalent bond connects the hydrogen atom with a nitrogen atom of the imidazoline ring, and wherein at least one hydrogen bond connects the hydrogen atom with the first oxygen (O) of the sulfur-based fragment and the second oxygen (O) of the sulfur-based fragment.

14. The stabilized imidazoline complex of claim 13, wherein the proton-stabilizing molecule is a dye selected from the group consisting of phenol red, pyrocatechol violet, eriochrome cyanine R, cresol red, chlorophenol red, bromocresol purple, bromophenol blue, bromothymol blue sodium salt, xylenol orange tetrasodium salt, xylenol orange disodium salt, methylthymol blue sodium salt, bromothymol blue, bromocresol green, and m-cresol purple.

15. The stabilized imidazoline complex of claim 13, wherein the positively charged imidazoline compound is derived from the reaction of a diethylenetriamine (DETA)- or aminoethylethanolamine (AEEA)-base compound with a fatty acid.

16. A method of preparing a stabilized imidazoline complex comprising the steps of:
obtaining a positively charged imidazoline compound; and
mixing the positively charged imidazoline compound with a proton-stabilizing molecule that comprises a substructure of SSS═(O═S1(═O)CCCCO1), wherein the proton-stabilizing molecule comprises a first lone-pair-bearing atom and a second lone-pair-bearing atom.

17. The method of claim 16, wherein the step of obtaining the positively charged imidazoline compound further comprises the step of protonating an unprotonated imidazoline to obtain the positively charged imidazoline compound.

18. The method of claim 16, further comprising the step of:
adding a pH modifier to promote complexation between the positively charged imidazoline compound and the proton-stabilizing molecule.

19. The method of claim 16, further comprising the steps of:
selecting a solvent that does not disrupt complexation between the positively charged imidazoline compound and the proton-stabilizing molecule; and
dissolving the proton-stabilizing molecule in the solvent.

20. The method of claim 16, further comprising the step of:
preparing the stabilized imidazoline complex in a blend with an inhibitor, where the inhibitor is a primary amine, a secondary amine, a tertiary amine, a quaternary amines, a sulfur-based compound, a phosphate ester, an ethoxylated imidazoline, an alkylpyridine, or a surfactant.
